(19) United States
(12) Patent Application Publication
(10) Pub. No.: US 2018/0318209 A1
(43) Pub. Date: Nov. 8, 2018

(54) FILM COMPOSITIONS OF SAMIDORPHAN AND BUPRENORPHINE

(71) Applicant: SUN PHARMACEUTICAL INDUSTRIES LIMITED, Mumbai (IN)

(72) Inventors: Pradeep Sanghvi, North Brunswick, NJ (US); Umesh PAI, Monroe, NJ (US)

(21) Appl. No.: 15/974,320

(22) Filed: May 8, 2018

(30) Foreign Application Priority Data
May 8, 2017 (IN) 201711016156

(51) Int. Cl. A61K 9/00; A61K 31/485; A61K 9/70

(52) U.S. Cl. CPC A61K 9/006; A61K 9/7007; A61K 31/485

(57) ABSTRACT

The present invention relates to a film for the oral transmucosal administration comprising therapeutically effective amount of a combination of samidorphan and buprenorphine or pharmaceutically acceptable salts thereof. The size of said film is about 0.2 to about 30 cm². The film compositions of the invention are useful for sublingual as well as buccal applications. The invention further relates to process of preparing said film compositions as well as method of treating major depressive disorder and opioid dependence by administering such film to the subject in need thereof.

### FIELD OF THE INVENTION

The present invention relates to a film composition for oral transmucosal administration comprising therapeutically effective amount of a combination of samidorphan and buprenorphine or pharmaceutically acceptable salts thereof. The size of said film is about 0.2 to about 30 cm². The film compositions of the invention are useful for sublingual as well as buccal applications. The invention further relates to a process of preparing said film compositions as well as method of treating major depressive disorder and opioid dependence by administering such film to the subject in need thereof.

### BACKGROUND OF THE INVENTION

Treatment resistant depression is a widespread disease where patients with major depressive disorders do not achieve an adequate response to existing therapies. Buprenorphine, a partial μ-opioid agonist, has been reported to be useful in treating treatment resistant depression in patients.

Samidorphan is an opioid antagonist (also known as 3-carboxamido-4-hydroxynaltrexone) that preferentially acts as an antagonist of the μ-opioid receptor. It is under development for the treatment of major depressive disorder and also under investigation for treatment of alcoholism and cocaine addiction.

The combination of buprenorphine and other μ-opioid antagonist naloxone is commercially available as buccal, sublingual film (Suboxone®) in US since 2010 and is indicated for the treatment of opioid dependence. Buprenorphine buccal film (Belbuca®) is also commercially available in US since 2015 and is indicated for around-the-clock treatment of moderate to severe chronic pain that is not controlled by other medicines.

U.S. Pat. No. 8,822,488 discloses a composition comprising buprenorphine and a μ-opioid receptor antagonist wherein the composition is characterized by an Agonist: Antagonist Activity Index (AAnAI) of between about 0.70 and about 2.2. It further relates to the treatment of depression by administering such composition to the subject in need thereof.

The fixed-dose combination of buprenorphine and samidorphan as sublingual tablets is under development for the treatment-refractory depression.

It is known from the literature that drug absorption from buccal and sublingual routes offers a number of benefits over other drug delivery approaches and allows drugs to circumvent some of the body's natural defense mechanisms like first pass metabolism, harsh stomach environment etc. Both the routes have high overall permeability as compared to other mucosae of the mouth.

There is currently a need for a dosage form such as orally dissolvable film that provides the desired therapeutic effect, while providing an adhesive effect in the mouth, rendering it difficult to remove once placed in mouth. The film dosage form is a great advantage especially for those patients who are mentally ill, disabled and uncooperative. Moreover, due to other advantages such as ease in handling, quick dissolution, faster onset and non-requirement of water for administration, the film dosage form would increase patient compliance.

The inventors of the present invention have developed a film composition for oral transmucosal administration comprising a therapeutically effective amount of a combination of samidorphan and buprenorphine or pharmaceutically acceptable salts thereof.

### SUMMARY OF THE INVENTION

The present invention relates to a film composition for oral transmucosal administration comprising therapeutically effective amount of a combination of samidorphan and buprenorphine or pharmaceutically acceptable salts thereof. The film compositions are useful for sublingual as well as buccal applications. The size of said film is about 0.2 to about 30 cm².

### DETAILED DESCRIPTION OF THE INVENTION

A first aspect of the present invention provides a film composition for oral transmucosal administration comprising therapeutically effective amount of a combination of samidorphan and buprenorphine or pharmaceutically acceptable salts thereof, wherein said film has a size of about 0.2 to about 30 cm².

The term “samidorphan” as used herein refers to Samidorphan base as well as other pharmaceutically acceptable salts.

The pharmaceutically acceptable salts of samidorphan include but not limited to salts prepared from pharmaceutically acceptable non-toxic acids or bases including inorganic acids and bases and organic acids and bases. Salts may be prepared from pharmaceutically acceptable non-toxic acids including inorganic and organic acids. Suitable pharmaceutically acceptable acid addition salts include acetic, benzenesulfonic (besylate), benzoic, camphorsulfonic, citric, ethenesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric acid and p-toluenesulfonic. In a particular embodiment, the salt of Samidorphan is L-malate.

The term “buprenorphine” as used herein refers to buprenorphine base as well as other pharmaceutically acceptable salts.

The pharmaceutically acceptable salts of buprenorphine include but not limited to hydrochloride, sulfate, citrate, acetate, trifluoroacetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucoronate, saccharate, formate, benzoate, glutamate, methanesulfonate, ethane sulfonate, benzene sulfonate, p-toluene sulfonate, and pamoate salts. Salts of Buprenorphine also include salts formed from organic or inorganic bases such as hydroxides of sodium, potassium, cesium, lithium; calcium, magnesium; aluminum, zinc; ammonia and organic amines, such as unsubstituted or hydroxy-substituted mono-, di-, or trialkylamines; dicyclohexylamine; tributyl amine; pyridine; picoline; N-methyl-N-ethylamine; diethylamine; triethylamine; mono-, bis-, or tris-(2-hydroxy-(C1-C3)alkyl amines), such as mono-, bis-, or tris-(2-hydroxyethyl)amine, 2-hydroxy-tri-butylamine, or tris-(hydroxymethyl)methylamine, N,N-di-[(C1-C3)alkyl]-N-(hydroxy-(C1-C3)alkyl)-amines, such as N,N-dimethyl-N-(2-hydroxyethyl)amine, or tri-(2-hydroxy ethyl)amine; N-methyl-D-glucamine; and amino acids such as arginine and lysine. In a particular embodiment, the salt of Buprenorphine is hydrochloride.

In one of the embodiments of above aspect, the film is for sublingual administration.

In yet another embodiment of above aspect, the film is for buccal administration.

In another embodiment of above aspect, samidorphan and buprenorphine are present in the weight ratio of 1:1.

In another embodiment of this aspect, the film for buccal and sublingual administration has a size of about 0.2 to about 20 cm².

In another embodiment of this aspect, the film for buccal and sublingual administration has a thickness in the range of about 50 to about 1500 μm.

In another embodiment of this aspect, the film for buccal and sublingual administration has a tensile strength in the range of about 0.05 to about 50 MPa.

In still another embodiment of this aspect, the film for buccal and sublingual administration has a folding endurance value in the range of about 25 to about 500.

In another embodiment of this aspect, the film for buccal and sublingual administration has in-vivo residence time of at least 10 minutes.

In another embodiment of this aspect, the film for buccal and sublingual administration has a drug loading of about 0.25 to about 10 mg/cm′.

In yet another embodiment of this aspect, samidorphan and buprenorphine have a substantially uniform distribution in the longitudinal and lateral directions of single layer.

In another embodiment of this aspect, the films for buccal and sublingual administration are stable for one month when stored at 40±2° C./75% RH.

In yet another embodiment of this aspect, the films for buccal and sublingual administration have no irritation potential for oral mucosa.

In yet another embodiment of this aspect, the films for buccal and sublingual administration have a drug uniformity content of about 85% to about 115%

In another embodiment of this aspect, the films for buccal and sublingual administration have a water content of about 2% to about 20%.

In another embodiment of this aspect, the films for buccal and sublingual administration have a surface pH of about 5 to about 7.

In another embodiment of this aspect, the films for buccal and sublingual administration exhibit a disintegration time of about 5 to about 120 seconds.

In another embodiment of this aspect, the films for buccal and sublingual administration exhibit a disintegration time of about 5 to about 25 seconds.

The term “disintegration” as used herein refers to a state in which any residue of the film remaining on the screen of the test apparatus is a soft mass having no palpably film core, or fragments of coating.

In yet another embodiment of this aspect, the films for buccal and sublingual administration have a swelling index of about 2 to about 10.

In yet another embodiment of this aspect, the films for buccal and sublingual administration have assay values of buprenorphine in the range of about 95 to about 100%.

In yet another embodiment of this aspect, the films for buccal and sublingual administration have assay values of samidorphan in the range of about 95 to about 100%.

A second aspect of the present invention provides a film for the oral transmucosal administration comprising therapeutically effective amount of a combination of samidorphan and buprenorphine or pharmaceutically acceptable salts thereof, wherein said film is stable in mucous enzymatic degradation as determined by in vitro stability studies carried out in presence of simulated saliva of pH 6.8.

A third aspect of the present invention provides a film for the oral transmucosal administration comprising therapeutically effective amount of a combination of samidorphan and buprenorphine or pharmaceutically acceptable salts thereof, wherein said film has a drug release profile comparable to sublingual tablet comprising said combination.

In one of the embodiments, the film provides an in-vivo plasma profile having a Cₘₐₓ value of about 0.5 to about 15 ng/ml for Buprenorphine.

In another embodiment of above aspect, the film provides an in-vivo plasma profile having a Cₘₐₓ value of 0.5 to about 40 ng/ml for Samidorphan.

A fourth aspect of the present invention provides a process for the preparation of film comprising samidorphan and buprenorphine or pharmaceutically acceptable salts comprising the steps of:
i) dissolving one or more mucoadhesive polymers in a suitable solvent system;
ii) dissolving or dispersing the active agents and other pharmaceutically acceptable excipients in a suitable solvent to form a solution;
iii) mixing the solutions obtained from steps i) and ii) and stirring it continuously so that no sedimentation is observed during the entire casting process and
iv) casting the resulting solution as a film followed by drying.

In one of the embodiments of this aspect, the wet film obtained is dried using controlled bottom drying as well as top drying so as to maintain the uniformity of the film composition.

In another embodiment of above aspect, the process for preparation of film comprising samidorphan and buprenorphine or acceptable salts comprises the steps of:
i) dispersing samidorphan in a suitable solvent;
ii) blending one or more mucoadhesive polymers and adding to solution of step i);
iii) adding buffering agents, flavours and sweetening agents into the above solution of step ii) to form a uniform mixture;
iv) adding buprenorphine and other pharmaceutically acceptable excipients into step iii) and
v) rolling the final solution and coating with polyester release liner followed by drying wherein the sequence of steps i) to step iv) can be changed.

Suitable solvents include aqueous as well as non-aqueous solvents such as alcoholic or hydro-alcoholic systems. Examples of solvents include, but are not limited to, methanol, ethanol, isopropanol, butanol, acetone, ethylacetate, cyclohexane, water and mixtures thereof.

A fifth aspect of the present invention provides a process for the preparation of film of samidorphan and buprenorphine or pharmaceutically acceptable salts by hot-melt extrusion comprising the steps of:
i) preparing the blend of samidorphan and buprenorphine with one or more other pharmaceutically acceptable excipients in dry state;
ii) melting the blend of step i) and forcing the molten mixture through an orifice so that homogenous material is obtained and
iii) casting the material obtained therefrom into a film.

In sixth aspect of the present invention, there is provided a buccal film for transmucosal administration comprising therapeutically effective amount of a combination of samidorphan and buprenorphine or pharmaceutically acceptable salts thereof, wherein said film comprises a non-adhesive impermeable layer superimposed on top of drug loaded mucoadhesive layer.

In one of the embodiments of this aspect, said non-adhesive impermeable layer is free of drug.

In another embodiment of this aspect, said non-adhesive impermeable layer comprises at least one drug.

In another embodiment of above aspect, said non-adhesive impermeable layer comprises Samidorphan.

In another embodiment of this aspect, said non-adhesive impermeable layer comprises water-soluble or water insoluble polymers.

In another embodiment of this aspect, said non-adhesive impermeable layer is distinguished from drug containing layer by addition of colors and opacifiers.

The term “non-adhesive impermeable layer” as used herein, refers to a layer or coating or barrier layer, capable of facilitating a unidirectional flux of drug in its direction and does not adhere to surfaces in the oral cavity. It protects the drug layer from salivary flow dissolution, thereby increasing the degree of drug available for buccal or sublingual absorption and decreasing the loss of drug to salivary distribution.

Examples of polymers that are used in non-adhesive impermeable layer include but not limited to hydroxyethyl cellulose, hydroxypropyl cellulose, hydroxyethylmethyl cellulose, polyethylene glycol, ethylene oxide-propylene oxide co-polymers, collagen and derivatives, polymethacrylates such as Eudragit® NE 40 D; albumin, polyaminoacids and derivatives, polyphosphazenes, polysaccharides and derivatives, chitin and chitosan or a combination thereof

In seventh aspect of the present invention, there is provided a method of treating a major depressive disorder and opioid abuse or addiction by buccal or sublingual administration comprising administering a film composition comprising samidorphan and buprenorphine or acceptable salts to a subject in need thereof.

The film composition of the present invention may further comprise other pharmaceutically acceptable excipients selected from the group consisting of mucoadhesive polymers, buffering agents, surfactants, permeation enhancers, plasticizers, saliva stimulating agents, flavoring agents, sweetening agents, coloring agents and mixtures thereof.

The mucoadhesive polymer may be selected from hydrophilic polymers and hydrogels.

Examples of mucoadhesive polymers include cellulose or cellulose derivatives such as carboxymethylcellulose, hydroxyethyl cellulose, hydroxypropyl cellulose, hydroxypropylmethyl cellulose and methyl hydroxyethyl cellulose, pullulan, polyvinyl alcohol, polyvinyl pyrrolidone, polyethylene glycol, polyethylene oxide, ethylene oxide-propylene oxide co-polymers, pectin, agarose, gelatin, hyaluronic acid, various gums such as guar gum, acacia gum, xanthum gum, tragacanth gum, gellan, carrageenan, pectin and sodium alginate, maltodextrin, methylmethacrylate copolymer, collagen and derivatives, starch and modified starch, gelatin, albumin, poly(acrylic acid)based polymers such as polymerized rosin, Kollicoat®, Lycoat® NG73, polyaminoacids and derivatives, polysaccharides and derivatives, chitin, cationic polymers such as chitosan and nonionic polymers such as Eudragit® analogues or mixtures thereof. The amount of mucoadhesive polymer may range from about 5% to about 50% by weight of the film composition.

Examples of buffering agents include, but not limited to citric acid, lactic acid, tartaric acid, maleic acid, fumaric acid, succinic acid and phosphoric acid. The buffering agents are added in sufficient amount to provide a desired local pH of about 3 and to ensure optimum absorption of drugs from the film compositions.

Examples of surfactants include, but not limited to, poloxamers such as poloxamer 407, poloxamer 124, poloxamer 188, poloxamer 237, poloxamer 338 and the like, sodium lauryl sulfate, benzalkonium chloride, benzethonium chloride, sorbitan monostearate, polyoxyethylene sorbitan fatty acid esters such as Polysorbate 20, Polysorbate 40, Polysorbate 60, and Polysorbate 80; non-ethoxylated glyceryl monostearate, cetomacrogol, cetostearyl alcohol, sodium stearoyl lactylate, lecithin, sorbitan fatty acid esters such as span 20, span 60, span 40 and span 80 and mixtures thereof. The amount of surfactant may range from about 0.05% to about 5% by weight of the film composition.

Examples of permeation enhancers include, but not limited to, surfactants including ionic such as sodium lauryl sulphate, sodium laurate, polyoxyethylene-20-cetylether, laureth-9, sodium dodecyl sulfate (SDS), dioctyl sodium sulfosuccinate, nonionic surfactants such as polyoxyethylene-9-Laurylether, tween 80, nonylphenoxypolyoxyethylene, polysorbates, sodium glycocholate; bile salts and derivatives such as sodium deoxycholate, sodium taurocholate, sodium taurodihydrofusidate (STDHF), sodium glycodihydrofusidate, sodium deoxycholate; fatty acids and derivatives such as oleic acid, caprylic acid, Lauric acid, linolein acid, acylcholines, acylcarnitine, sodium caprate, chelating agents such as EDTA, citric acid, salicylates; sulfoxides such as dimethyl sulfoxide, decylmethyl sulfoxide; polyols such as propylene glycol, polyethylene glycol, glycerol, propanediol; monohydric alcohols such as ethanol, isopropanol; non-surfactants such as unsaturated cyclic ureas, azone (1-dodecylazacycloheptan-2-one) (laurocapram), cyclodextrin, enamine derivatives, terpenes, liposomes, acyl carnitines, cholines and mixtures thereof. The amount of permeation enhancer may range from about 0.01 to about 5% by weight of the film composition.

Examples of plasticizers include, but not limited to, glycerol, sorbitol, propylene glycol, polyethylene glycols such as PEG 400, PEG 200, PEG 600, triacetin, dimethyl phthalate, di-butylphthalate, dibutyl sebacate, distilled acetylated monoglycerides, triethyl citrate, acetyl citrate castor oil, acetyl triethyl citrate and other citrate esters. These are present in the range from about 1% to about 20% of the weight of the film composition. In a preferred embodiment, the plasticizer used in the film composition includes polyethylene glycol 400.

Examples of saliva stimulating agents include, but not limited to, citric acid, malic acid, lactic acid, ascorbic acid, malic acid, adipic acid, fumaric acid, tartaric acid and combinations thereof. These are present in the range from about 2% to about 10% by weight of the film composition. In a preferred embodiment, the saliva stimulating agent includes citric acid.

Examples of flavors include, but not limited to, menthol, peppermint, essential oils such as menthol, methyl salicylate, eucalyptol, thymol, vanilla, intense mints e.g. peppermint, sweet mint, spearmint, wintergreen, cinnamon, clove, sour fruit flavor such as lemon, orange or sweet confectionary flavors such as vanillin, chocolate, or fruit essence e.g. apple, raspberry, cherry and pineapple and combinations thereof. These are present in the range from about 2% to about 10% w/w by weight of the film composition.

Examples of sweetening agents include, but not limited to, natural and artificial sweeteners. The water-soluble sweetening agents are selected from monosaccharides, disaccharides and polysaccharides such as xylose, ribose, glucose (dextrose), mannose, galactose, fructose (levulose), sucrose (sugar), maltose, invert sugar (a mixture of fructose and glucose derived from sucrose), partially hydrolyzed starch, corn syrup solids, dihydrochalcones, monellin, steviosides, and glycyrrhizin; water-soluble artificial sweeteners such as the soluble saccharin salts, i.e., sodium or calcium saccharin salts, cyclamate salts, acesulfame-K, the free acid form of saccharin; dipeptide based sweeteners, such as L-aspartic acid derived sweeteners, aspartame, sucralose; other protein based sweeteners and mixtures thereof.

The coloring agents may be selected from any FDA approved colors for oral use. Coloring agents may be selected from natural food colors and dyes suitable for food, drug and cosmetic applications (also known as FD&C dyes and lakes). Examples of coloring agents include, but not limited to, FD &C Yellow 5, FD &C Yellow 6, FD&C Blue 1, FD&C Blue 2, FD&C Red 1, FD&C Red 2, FD&C Red 3, FD&C Red 40, FD&C orange, FD&C Green 3 and mixtures thereof. In the preferred embodiment, the coloring agent includes FD &C Yellow 6.

The sublingual or buccal film of samidorphan and buprenorphine or pharmaceutically acceptable salts is packaged in unit dose blister packs, pouches in a carton, foil, paper, vials with screw or flip-top lids, bottles with screw or flip-top lids, or any other convenient package form. The package is temper-resistant and has a high degree of environmental protection.

The term “about” as used herein, refers to any value which lies within the range defined by a variation of up to ±10% of the value.

The term “film” includes thin films and sheets, in any shape, including rectangular, square, circle, ellipse, triangle, polygon or any other desired shape.

The term “drug” as used herein includes samidorphan and buprenorphine or pharmaceutically acceptable salts thereof.

The term “oral transmucosal administration” refers to administration of drug(s) through the oral mucosa to achieve systemic effects.

The term “mucoadhesive”, as used herein, refers to a material that adheres to a mucosal tissue surface in-vivo and/or in-vitro upon hydration. Such adhesion will adherently localize the dosage form onto the mucus membrane and requires the application of a force of at least about 50 dynes/cm′ to separate the mucoadhesive material from the mucus membrane.

Bioequivalence is established by comparing pharmacokinetic parameters, for example mean AUC and Cₘₐₓ of film of the present invention with sublingual tablets comprising said combination in healthy human subjects.

The term “AUC” refers to the area under the time/plasma concentration curve after administration of film of Samidorphan and buprenorphine.

The term “Cₘₐₓ” refers to the maximum concentration of samidorphan and buprenorphine in blood following the administration of film.

The following examples represent various embodiments according to the present invention. The examples are given solely for the purpose of illustration and are not to be construed as limitations of the present invention, as many variations thereof are possible without departing from the spirit and scope of the invention.

### EXAMPLES

### Example 1

| Ingredients | Amount (in mg) |
|---|---|
| Buprenorphine Hydrochloride | 2.00 |
| Samidorphan | 2.00 |
| HPMC K4M | 0.50 |
| Polyethylene glycol 400 | 0.25 |
| Citric acid | 0.20 |
| Dibutyl phthalate | 0.15 |
| Flavors | 0.15 |
| Sodium lauryl sulfate | q.s. |
| Titanium dioxide | q.s. |
| Purified Water | q.s. |

### Procedure:

1. Dissolve HPMC and polyethylene glycol 400 in purified water.
2. Dissolve samidorphan and buprenorphine hydrochloride along with sodium lauryl sulphate, citric acid, titanium dioxide, dibutyl phthalate and flavors in water to form a homogenous solution.
3. Mix the above formed solutions of steps 1 & 2 and stir continuously so that no sedimentation is observed during the entire casting process.
4. Cast the resultant solution from step 3 as a film followed by drying.

### Example 2

| | | |
|---|---|---|
| Ingredients | Quantity (in mg) | % w/w |

Solution 1

| | | |
|---|---|---|
| Polyethylene Oxide NF (Sentry Polyox | 4.500 | 7.76% |
| WSR N10 - Grade) | | |
| Polyethylene Oxide NF (Sentry Polyox | 6.500 | 11.21% |
| WSR N80 - Grade) | | |
| Hydroxypropyl methylcellulose K4M | 0.500 | 0.86% |
| Polyethylene Glycol 400 NF | 1.000 | 1.72% |
| Samidorphan | 2.000 | 3.45% |
| Ethanol *² | 10.000 | 17.24% |
| Ethanol *² | 3.000 | 5.17% |

Solution 2

| | | |
|---|---|---|
| Buprenorphine hydrochloride USP *³ | 2.156 | 3.72% |
| Anhydrous Citric Acid USP/Citric | 1.200 | 2.07% |
| Acid Anhydrous Ph. Eur. | | |
| Dibutyl Sebacate NF | 0.140 | 0.24% |
| (QD 04) N and A Lemon FL # 11057 *¹ | 0.500 | 0.86% |
| Sucralose NF | 1.500 | 2.59% |
| FD&C Yellow 6 | 0.010 | 0.01% |
| Purified Water (for dissolving) *² | 20.000 | 34.48% |
| Purified Water (for rinsing)- *² | 5.000 | 8.62% |
| TOTAL (g) | 58.006 | 45.973 |
| Solvent (g) | 38.000 | 29.640 |
| Net (g) | 20.006 | 16.333 |
| % Solid Content | 34.49% | 35.53% |

*¹ Quantity of (QD04) N and A Lemon FL # 11057 calculated considering 65% water content which is to be evaporated during process.
*² Processing solvent evaporates during process and does not remain present in final product except in traces.
*³ Quantity based on 100% assay. Input per batch to be calculated based on actual assay.

### Manufacturing Process:

### Preparation of Solution 1

1. Samidorphan was dispersed in ethanol.
2. Polyox WSR N10 and Polyox WSR N80 and Hydroxypropyl methylcellulose K4M were blended together.
3. The blend obtained from step 2 was dispersed in ethanol.
4. Polyethylene glycol 400 was added into the dispersion of step 3.

### Preparation of Solution 2

5. Citric acid was added to purified water and stirred until it is completely dissolved.
6. Sucralose and lemon flavor were added into the solution of step 5.
7. Dibutyl sebacate was added into solution of step 6.
8. Buprenorphine hydrochloride was added into solution of above step and mixed until a uniform dispersion was obtained.
9. The coloring agent FD&C Yellow 6 was added to dispersion of step 8.

### Preparation of Final Dispersion

10. The dispersion of step 4 was slowly added to dispersion of step 9.
11. The containers were rinsed with purified water and ethanol and the rinsed solution was added to step 10.
12. The final dispersion was stirred for 10 minutes at 500 rpm and rolled overnight.

### Film Coating

13. The final dispersion obtained after rolling was coated on a polyester (PET) release liner and dried.

The films obtained according to Example 2 were subjected to different tests and the results obtained from these tests are summarized in below tables:

TABLE 1
Determination of physical characteristics:
Description (Visual):

| Bulk solution | Final film |
|---|---|
| (after overnight rolling) | (after coating and drying) |
| Saturated orange with no air | Pale, translucent, orange colored |
| bubbles. | film |

### Thickness of Film:

The thickness of the film prepared according to Example 2 was measured using digital micrometer.

The resulting thickness was found to be 5.0 mils (=127 microns).

### Water Content:

The water content was checked by using Karl Fischer apparatus.

The pre-weighed film sample was-put in the apparatus and titrated with Karl Fischer reagent to the electrometric end-point. The percentage of water content obtained by Karl Fisher titrator was recorded

Moisture content %=Initial weight−Final weight/Initial weight×100

The water content of the film of Example 2 was calculated to be 3.25%

### Size/Area of the Film:

The films of Example 2 were cut into dimensions such that each film weigh 2 mg of Buprenorphine and 2 mg of Samidorphan and cross sectional area of the film was determined. The average size of the film was obtained to be 13.0×12.8 mm (=16.64 cm²)

### Surface pH:

The film of Example 2 was allowed to swell by keeping it in contact with 1 ml of purified water for 10 minutes at room temperature and pH was noted using pH meter by bringing the electrode in contact with the surface of the film, allowing it to equilibrate for 1 minute.

The pH value of the surface of the film was found to be 3.99. The surface pH in the range 5.6 to 7.4 confirmed the compatibility of the films with oral mucosa and no risk of mucosal damage or irritation on usage.

### Assay:

The sample films of Example 2 were dissolved in a suitable solvent and analyzed by HPLC system equipped with PDA detector and suitable data collection system. The column temperature was set at 50° C. with the flow rate in the system as 1.5 ml/min The assay values of Buprenorphine was found to be 99.1% and Samidorphan was found to be 99.9%

### Drug Content Uniformity:

The uniformity of drug content was determined by dissolving the film in a suitable solvent and making up the volume to 100 ml in volumetric flask. Then a sample of 1 ml of this solution was withdrawn and diluted to 10 ml with a suitable solvent. The absorbance of this solution was measured using UV visible spectrophotometer and the concentration was-calculated. By calculating the dilution factor, the drug content was calculated. The average of 10 samples of the film composition of Example 2 were taken and was found to be within the standard limits of 85-115%. The standard deviation was calculated and results are given in Tables 2 and 3.

TABLE 2
Content uniformity for Buprenorphine

| Sample No. | Film weight (mg) | % Content Uniformity |
|---|---|---|
| 1 | 20.24 | 96.5 |
| 2 | 20.03 | 97.3 |
| 3 | 20.00 | 98.0 |
| 4 | 20.15 | 98.5 |
| 5 | 20.38 | 97.5 |
| 6 | 20.37 | 99.9 |
| 7 | 20.46 | 100.8 |
| 8 | 20.32 | 100.6 |
| 9 | 20.08 | 98.7 |
| 10 | 20.14 | 100.1 |
| Mean | 20.22 | 98.79 |
| Max | 20.46 | 100.80 |
| Min | 20.00 | 96.50 |
| Std. Dev | 0.16 | 1.49 |

TABLE 3
Content uniformity for Samidorphan

| Sample No. | Film weight (mg) | % Content Uniformity |
|---|---|---|
| 1 | 20.24 | 97.4 |
| 2 | 20.03 | 98.4 |
| 3 | 20.00 | 98.9 |
| 4 | 20.15 | 99.4 |
| 5 | 20.38 | 98.4 |
| 6 | 20.37 | 100.8 |
| 7 | 20.46 | 101.6 |
| 8 | 20.32 | 101.5 |
| 9 | 20.08 | 99.6 |
| 10 | 20.14 | 100.7 |
| Mean | 20.22 | 99.67 |
| Max | 20.46 | 101.60 |
| Min | 20.00 | 97.40 |
| Std. Dev | 0.16 | 1.43 |

### Tensile Strength and Percent Elongation:

The tensile strength of the film was determined using Instron 5943 material testing system with 50N load cell. The tensile strength of film of Example 2 was determined by applying the maximum stress to a point till the film ruptured and was computed as a mean of 5 sample measurements.

Young's modulus was calculated as the ratio of applied stress over strain in the region of elastic deformation and results of these calculated parameters are shown in Table 4.

TABLE 4
Tensile strength and percent elongation of different film samples
of Example 2

| | Tensile Strain | |
|---|---|---|
| | (Extension) at Break | Tensile Stress at break |
| | (Standard) | (Standard) |
| Sample No. | (% Elongation) | (MPa) |
| 1 | 87.34603 | 0.08347 |
| 2 | 98.12536 | 0.12072 |
| 3 | 95.80032 | 0.16161 |
| 4 | 74.55987 | 0.25209 |
| 5 | 124.93948 | 0.13849 |
| Average | 96.15421 | 0.151276 |
| Std Dev | 18.551 | 0.063 |

| | |
|---|---|
| Young's Modulus | $\frac{Stress}{Strain} = \frac{0.151276 MPa}{0.9615421} = 0.157326 .445 MPa = 157 , 326.445 N \text{/} {m}^{2}$ |

### Folding Endurance:

It was calculated by manual folding a small strip of film at the same place several times until it ruptured. The number of times the film was folded without breaking provided the folding endurance value. An average of 5 measurements was taken and results are provided in Table 5.

TABLE 5
Folding endurance of different samples film of Example 2

| Film No. | Folding Endurance Value |
|---|---|
| 1 | 71 |
| 2 | 80 |
| 3 | 97 |
| 4 | 85 |
| 5 | 70 |
| Average | 81 |

### Swelling Index:

The swelling studies of films were conducted in simulated salivary fluid of pH 6.8. The film sample accurately weighed by using digital weighing balance (W1) and placed in simulated salivary medium contained in a petri dish. At definite time intervals (30, 60, 90, 120, 150, 180, 210 seconds), films removed and blotted between filter paper and reweighed (W2). The swelling index was calculated by the following formula and results of an average of 3 sample measurements are provided in table 6:

S·I=W2−W1/W1×100

TABLE 6
Swelling index studies of different sample films of Example 2

| Film No. | Swelling Index |
|---|---|
| 1 | 3.95 |
| 2 | 3.00 |
| 3 | 3.26 |
| Average | 3.40 |

### Disintegration Test:

Disintegration times were measured in vitro for six samples by the standard United States Pharmacopeia (USP) disintegration method 1000 ml purified water at 37° C. using the Distek 3100 USP disintegration apparatus. The time required for full disintegration was recorded for each film and the results are provided in the Table 7.

TABLE 7
Disintegration time of films

| Film No. | Disintegration Time | |
|---|---|---|
| 1 | 1 min 45 seconds | |
| 2 | 19 | seconds |
| 3 | 54 | seconds |
| 4 | 15 | seconds |
| 5 | 1 | min |
| 6 | 1 min 20 seconds | |
| Average | 56 | seconds |

### Dissolution Test:

In vitro dissolution test was carried out according to the USP II (paddle over disk) dissolution apparatus using 300 ml of simulated saliva of pH 6.8 as dissolution medium at a temperature of 37° C. and 100 rpm. Three film sample measurements of Example 2 were taken and samples were withdrawn at time intervals of 2, 3, 5, 7, 10, 15, 20 and 30 minutes. The absorbance was measured using UV-visible spectrophotometer and percent drug release was calculated and results are given in Tables 8 and 9.

TABLE 8
In-vitro dissolution data for Buprenorphine

| Sample | 2.0 | 3.0 | 5.0 | 7.0 | 10.0 | 15.0 | 20.0 | 30.0 |
|---|---|---|---|---|---|---|---|---|
| No | min | min | min | min | min | min | min | min |
| 1 | 63 | 73 | 77 | 80 | 81 | 82 | 83 | 84 |
| 2 | 69 | 75 | 79 | 80 | 83 | 84 | 83 | 83 |
| 3 | 69 | 76 | 78 | 77 | 81 | 81 | 81 | 81 |
| Mean | 67 | 75 | 78 | 79 | 82 | 82 | 82 | 83 |
| Max | 69 | 76 | 79 | 80 | 83 | 84 | 83 | 84 |
| Min | 63 | 73 | 77 | 77 | 81 | 81 | 81 | 81 |
| % RSD | 5.6 | 2.5 | 1.7 | 2.6 | 1.0 | 1.7 | 1.4 | 1.8 |

TABLE 9
In-vitro dissolution data for Samidorphan

| Sample | 2.0 | 3.0 | 5.0 | 7.0 | 10.0 | 15.0 | 20.0 | 30.0 |
|---|---|---|---|---|---|---|---|---|
| No. | min | min | min | min | min | min | min | min |
| 1 | 79 | 87 | 91 | 94 | 97 | 97 | 97 | 98 |
| 2 | 83 | 89 | 96 | 95 | 97 | 98 | 98 | 98 |
| 3 | 83 | 89 | 93 | 95 | 96 | 97 | 97 | 97 |
| Mean | 82 | 88 | 93 | 95 | 97 | 97 | 97 | 98 |
| Max | 83 | 89 | 96 | 95 | 97 | 98 | 98 | 98 |
| Min | 79 | 87 | 91 | 94 | 96 | 97 | 97 | 97 |
| % RSD | 2.9 | 1.3 | 1.2 | 0.5 | 0.5 | 0.5 | 0.6 | 0.4 |

### Stability Studies:

To assess the drug and formulation stability, the sublingual or buccal film was subjected to stability studies according to ICH guidelines. The formulated films were wrapped in aluminum foil and stored at temperatures of 40±2° C./75% RH for period of 1 month. The films were also tested at refrigerated temperature for 48 hour.

We claim:

1. A film composition for oral transmucosal administration comprising: a therapeutically effective amount of a combination of samidorphan and buprenorphine, or each as pharmaceutically acceptable salts thereof, wherein the size of said film is about 0.2 to about 30 cm².

2. The film composition according to claim 1, wherein the said film is for sublingual and buccal administration.

3. The film composition according to claim 1, wherein samidorphan and buprenorphine are present in the weight ratio of 1:1.

4. The film composition according to claim 1, wherein the said film has a thickness in the range of about 50 to about 1500 μm.

5. The film composition according to claim 1, wherein said film has a disintegration time of about 5 to about 120 seconds.

6. The film composition according to claim 1, wherein said film has a water content of about 2 to about 20%.

7. The film composition according to claim 1, wherein said film has a surface pH of about 5 to about 7.

8. The film composition according to claim 1, wherein said film has a drug uniformity content of about 85 to about 115%.

9. The film composition according to claim 1, wherein said film has a tensile strength of about 1 to about 50 MPa.

10. The film composition according to claim 1, wherein said film has a folding endurance of about 25 to about 500.

11. The film composition according to claim 1, wherein said film has a swelling index of about 2 to about 10.

12. The film composition according to claim 1, wherein said film has no irritation potential for oral mucosa.

13. The film composition according to claim 1, wherein said film further comprises at least one or more pharmaceutically acceptable excipients.

14. A film for the oral transmucosal administration comprising therapeutically effective amount of a combination of samidorphan and buprenorphine or each as pharmaceutically acceptable salts thereof, wherein said film is stable in mucous enzymatic degradation as determined by in vitro stability studies carried out in presence of simulated saliva of pH 6.8.

15. A buccal film for the transmucosal administration comprising therapeutically effective amount of a combination of samidorphan and buprenorphine or each as pharmaceutically acceptable salts thereof, wherein said film comprises a non-adhesive impermeable layer superimposed on top of drug loaded mucoadhesive layer.

16. The film composition according to claim 15, wherein said non-adhesive impermeable layer comprises water-soluble or water insoluble polymers.

17. The film composition according to claim 1, wherein said film is prepared by a process comprising the steps of:
i) dispersing Samidorphan in a suitable solvent to form a solution;
ii) blending one or more mucoadhesive polymers and adding to the solution of step i);
iii) adding buffering agents, flavours and sweeting agents into above solution of step ii) to form a uniform mixture;
iv) adding Buprenorphine and other pharmaceutically acceptable excipients into step iii);
v) rolling the final solution and coating with polyester release liner followed by drying; wherein the sequence of steps i) to step iv) can be changed.

18. The film composition according to claim 14, wherein said film is prepared by a process comprising the steps of:
i) dispersing Samidorphan in a suitable solvent;
ii) blending one or more mucoadhesive polymers and adding to the solution of step i);
iii) adding buffering agents, flavours and sweeting agents into above solution of step ii) to form a uniform mixture;
iv) adding Buprenorphine and other pharmaceutically acceptable excipients into step iii);
v) rolling the final solution and coating with polyester release liner followed by drying; wherein the sequence of steps i) to step iv) can be changed.

19. The buccal film composition according to claim 15, wherein said film is prepared by a process comprising the steps of:
i) dispersing Samidorphan in a suitable solvent;
ii) blending one or more mucoadhesive polymers and adding to the solution of step i);
iii) adding buffering agents, flavours and sweeting agents into above solution of step ii) to form a uniform mixture;
iv) adding Buprenorphine and other pharmaceutically acceptable excipients into step iii);
v) rolling the final solution and coating with polyester release liner followed by drying; wherein the sequence of steps i) to step iv) can be changed.

20. A method of treating a major depressive disorder and opioid abuse or addiction by buccal or sublingual administration comprising the step of: administering a film composition according to claim 1.
